(19) United States
(12) Patent Application Publication
(10) Pub. No.: US 2023/0298725 A1
(43) Pub. Date: Sep. 21, 2023

(54) BREATHING EXERCISE DEVICE

(71) Applicant: ZEPH TECHNOLOGIES INC., New York, NY (US)

(72) Inventors: Sevag Babikian, Beirut (LB); Rohan Manjanatha, Thousand Oaks, CA (US); John Barry, Philadelphia, PA (US)

(21) Appl. No.: 18/121,522

(22) Filed: Mar. 14, 2023

Related U.S. Application Data
(60) Provisional application No. 63/320,079, filed on Mar. 15, 2022.

(51) Int. Cl. G16H 20/30; G16H 40/67; G06Q 50/00

(52) U.S. Cl. CPC G16H 20/30; G16H 40/67; G06Q 50/01

(57) ABSTRACT

Disclosed are example embodiments of a breathing exercise device. The breathing exercise device including a respiratory muscle training (RMT) device. The RMT device configured to communicatively connect to an electronic communication device. The RMT device further configured to measure biometric data of a user and transmit the biometric data of the user to the electronic communication device. The biometric data is transmitted while streaming at least one of instructional video content and instructional audio content to the electronic communication device.

### CLAIM OF PRIORITY UNDER 35 U.S.C. § 119

The present application for patent claims priority to Provisional Application No. 63/320,079 entitled “BREATHING EXERCISE DEVICE” filed Mar. 15, 2022, and assigned to the assignee hereof and hereby expressly incorporated by reference herein.

### TECHNICAL FIELD

The disclosure relates generally to the field of fitness equipment and methods as well as respiratory rehabilitation and methods, and specifically and not by way of limitation, some embodiments are related to a system and method for measuring and synthesizing biometric data and providing exercise training instruction and classes.

### BACKGROUND

Humans are creatures of habit. Trying to change behavior is very difficult especially when it's something that is important, but not necessarily enjoyable. Humans are also narrative creatures, constantly seeing themselves by weaving together the past, present, and future in the form of goals and expectations. Allowing users to see how they have performed and are performing in an orderly manner helps emotionally compartmentalize and encourage progress. Existing respiratory muscle training (RMT) devices for fitness and health rehabilitation frequently lack core features such as feedback loops and require various disparate devices, making the user experience poor for building long-lasting habits that transform a respiratory exercise they must do into a way of life.

Formal Pulmonary Function Tests (PFTs) have been performed in hospitals since the 1920s to measure lung volume, capacity, rates of flow, and gas exchange of patients' lungs. Because the PFT is performed by a professional technician, this event must take place at a specific time and location. As a result, appointments might be unavailable or expensive if done prophylactically and not due to a medical pathology. Furthermore, during pandemic outbreaks, people may not want to go to in person appointments or appointments may be completely unavailable or reserved for only a small percentage of people who are patients.

While some existing at-home pulmonary tracking devices such as Powerbreathe [https://www.powerbreathe.com/] and training equipment such as Airofit [https://www.airofit.com/] use digital spirometry, display screens and mobile applications while a user trains, these systems lack a vocabulary, and uniform metric for users to easily understand the user's data and see how the user's data is being applied to their future trainings. Similarly, the exercise training programs lack an ability to engage the user in a way that builds a positive ritual or long-term routine. Existing RMT tracking and training devices use the traditional passive method of exercise training that allows users to select their own resistance. Without a trainer or technician present, this can create user compliance issues.

Existing at-home pulmonary tracking devices also overlook the cardiovascular connection with breathing and respiratory training, which is essential for improving a user's performance, providing more precise feedback, and understanding how the various body functions work together. There are devices which measure heart rate variability and heart rate EliteHRV [https://elitehrv.com/], Whoop [https://www.whoop.com/], but they are wearables and do not take any in-depth pulmonary readings. Furthermore, there is no at-home system that combines data into a simple metric to give users a better understanding of the complex interplay between heart, lungs, body, and mind. Moreover, none of the prior art RMT devices have the capability of collecting user data that enables users to review their progress and compare their results with the RMT training community, for example, by age group, by training activity, or goals.

Prior art devices such as Calm, Headspace, or Revert give users techniques for managing the mind, but there are ways to take it further and ensure users are using the right muscles to achieve these powerful effects over the autonomic nervous system. When the mind isn't where we want it to be, we can use the body to get to where we want the mind.

Accordingly, a need exists for an improved training device. Some embodiments may address the problems discussed above, e.g., using an RMT trainer device that may connect to a mobile application, e.g., via Bluetooth, to read and send biometric data while streaming instructional content and audio with social competition capabilities. Unlike prior ideas, some embodiments may be a complete system for easily understanding the cardio-pulmonary function in one simple metric and using a host of sensors to create a tailored respiratory workout program with adaptive resistance training and more in-depth feedback. Some embodiments may allow users to review their data within the context of their community and other users with similar goals, rather than just allowing users to see a comparison of their data based on a user's age-matched average.

### SUMMARY

In one example implementation, an embodiment may use an RMT trainer device that may connect to a mobile application, e.g., via Bluetooth, to read and send biometric data while streaming instructional content.

Disclosed are example embodiments of a breathing exercise device. The breathing exercise device including a respiratory muscle training (RMT) device. The RMT device configured to communicatively connect to an electronic communication device. The RMT device further configured to measure biometric data of a user and transmit the biometric data of the user to the electronic communication device. The biometric data is transmitted while streaming at least one of instructional video content and instructional audio content to the electronic communication device.

The features and advantages described in the specification are not all-inclusive. In particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating various examples embodiments of an exercise training and testing system that may include an RMT training device with a wireless connection to a smartphone with an RMT mobile application in accordance with the systems and methods described herein.

FIG. 2 is another diagram illustrating various examples embodiments of an exercise training and testing system that may include an RMT training device with a wireless connection to a smartphone with an RMT mobile application in accordance with the systems and methods described herein.

FIG. 3 is a diagram illustrating various examples embodiments of an inside of an RMT training device in accordance with the systems and methods described herein.

FIG. 4 is another diagram illustrating various examples embodiments of an inside of an RMT training device include a pressure sensor, a heart rate monitor, digital and local storage on the device, a communication device, a camera, and a microphones in accordance with the systems and methods described herein.

FIG. 5 is another diagram illustrating various examples embodiments of an inside of an RMT training device in accordance with the systems and methods described herein.

FIG. 6 is a diagram illustrating an RMT training device carrier in accordance with the systems and methods described herein.

FIG. 7 is a diagram illustrating various examples of the embodiment of the mobile interface which interacts with the RMT trainer device may communicate with the user interface via Bluetooth or Wi-Fi. Bluetooth (BLE) uses very little power to transfer data and instructions between the device and mobile phone in accordance with the systems and methods described herein.

FIG. 8 is another diagram illustrating various examples of the embodiment of the mobile interface which interacts with the RMT trainer device may communicate with the user interface via Bluetooth or Wi-Fi. Bluetooth (BLE) uses very little power to transfer data and instructions between the device and mobile phone in accordance with the systems and methods described herein.

FIG. 9 is another diagram illustrating various examples of the embodiment of a mobile application having an example mobile interface in which interacts with the RMT trainer device may communicate with the user interface via Bluetooth or Wi-Fi. Bluetooth (BLE) uses very little power to transfer data and instructions between the device and mobile phone in accordance with the systems and methods described herein.

FIG. 10 is another diagram illustrating a mobile application in accordance with the systems and methods described herein.

FIG. 11 is a diagram illustrating a start screen for a breathability test that may provide a single metric that weighs the different input data points at various weights to output a comprehensive Breathability fitness score accordance with the systems and methods described herein.

FIG. 12 is a diagram illustrating a breathability baseline test that may serve as a single, simple encompassing indicator of three central systems of the body in accordance with the systems and methods described herein.

FIG. 13 is a diagram illustrating a breathability baseline test as part of an onboarding experience to build the user profile in accordance with the systems and methods described herein.

FIG. 14 is a diagram illustrating a breathability score in accordance with the systems and methods described herein.

FIG. 15 is a diagram illustrating training in accordance with the systems and methods described herein.

FIG. 16 is another diagram illustrating a mobile application in accordance with the systems and methods described herein.

FIG. 17 is a diagram illustrating a summary page on a mobile application in accordance with the systems and methods described herein.

FIG. 18 is a diagram illustrating that the system follows a well-established IoT paradigm whereby the device is paired with a user account connected via Bluetooth low energy (BLE) or Wi-Fi to an app on the mobile device in accordance with the systems and methods described herein.

FIG. 19 is another diagram illustrating that the system follows a well-established IoT paradigm whereby the device is paired with a user account connected via Bluetooth low energy (BLE) or Wi-Fi to an app on the mobile device in accordance with the systems and methods described herein.

FIG. 20 is a diagram illustrating a summary of data and calculated scores that may be part of a breathability test on a mobile application in accordance with the systems and methods described herein.

FIG. 21 is a diagram illustrating an example needle value in accordance with the systems and methods described herein.

FIG. 22 is a diagram illustrating an example needle value in an open position in accordance with the systems and methods described herein.

FIG. 23 is a diagram illustrating an example needle value in a closed position in accordance with the systems and methods described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Various embodiments may include a system of breath training and exercise sessions that seek to train the breath and pulmonary system through Inspiratory Muscle Training (IMT) and Expiratory Muscle Training (EMT) protocols. The combination of these two protocols is referred to as Respiratory Muscle Training (RMT). Using an RMT device, also referred to herein as an RMT trainer, may allow the user to strengthen accessory lung muscles through a protocol of gradual applied airway resistance. The handheld RMT trainer acts as the engagement point for users to connect to instructor-led exercises and breath trainings either live, pre-recorded, or animated from any location. Whether remote or in person, trainings, and tests with the RMT trainer may collect cardiopulmonary biomarkers to aggregate a holistic picture of a user's general pulmonary fitness as they progress through trainings. In various embodiments, training programs may be designed with both active and passive resistance load controlling to enhance the training experience for more precise training and efficient results. For example, a combination of sensors, cameras, motors, and/or actuators may work together to allow some embodiments to accurately test and adjust resistances to provide effective training and outcomes.

In other embodiments, the content creation and content consumption between users and trainers may be dynamic and interactive. Plans created may be performed independently or within a group or class real-time or overtime. Qualified users may create their own plan with multiple sessions for other users to follow and participate live with one another, archived or live with their RMT trainers. Some embodiments support these networks of RMT exercises within a central system for digital storage and database functions that may be accessed from anywhere connectible to the internet. Some embodiments of an example RMT device may use one or more cameras, text, imagery, haptic motors, and lights to guide animated, live or archived exercise training sessions to ensure user compliance and proper form.

In various embodiments, the software may serve as an assessment and intervention solution. The RMT training device in the preferred embodiment is a handheld device that may connect via a network (e.g., Wi-Fi, Bluetooth, 5G) to a central processing system and database to test the user's cardiopulmonary performance and assess their pulmonary function. Some embodiments may allow for analysis of this intake data and for subsequent testing data to be stored as a database and later used for machine learning and/or for comparison purposes using a leaderboard of results or similar output comparison measures. In various embodiments, all the relevant input data may be calculated and computed into a single baseline score called the Breathability score. The Breathability metric may serve to guide users, e.g., as the “North Star” for users to assess the state of their cardio-pulmonary fitness and understand the progress they make while they train. Explainable machine learning will ensure that for example, if the user wanted to understand their Breathability score or training data further, an analysis is available to detail why a plan may have shifted, for example, to a higher resistance or a different track. Just as a personal trainer will observe injuries or new needs and adjust programming, accordingly, so will some embodiment's training features. In various embodiments, an algorithm of the user's age-matched results combined with the machine learning recommendation, will place them on a recommended training path with others identified as in the same age group, class, and goals. In various embodiments, the trainings may be designed to be a personalized path or ad-hoc exercises from the library of trainings.

In various embodiments, training programs may be uploaded to the central server and stored as a library. When a user chooses to join and watch the training programs through their phone or computer or tv, they may access the library over the internet and have the video play locally on their phone and the parameters of the exercise resistance sent to their RMT device. Predefined professional fitness options may be provided to create bespoke training programs with the goal of maximizing outcomes and optimizing efficient training. In use, the RMT device allows users to join live, archive remote training sessions where they may participate independently or in a class format within a closed or open group of other RMT trainers. Leaderboard display on the user's mobile device, for example, may be used to provide the users with an understanding how they are doing, how they are ranked, and provide motivation for continued training.

Once the predetermined plan has been completed, there is a platform of exercises available to the user to select created by celebrity athletes, yogis, breath coaches and other partnered content creators. Additionally, in other embodiments, Advanced users and Specialist users may also use the platform as a space to upload their breathing exercises for specific classes or for their private classes and groups. All categories of exercises will be available in the library for users to do after or during their personalized trainings.

Thus, it may be determined that some embodiments encompass methods and apparatus which allow for pulmonary function testing, breath training, increasing parasympathetic tone for anxiety and sleep, content creation, content consumption, content management, data management, data analysis and comparison. Some embodiments and potential interactions amongst all these processes will now be described in more detail.

Handheld RMT Trainer

How the Trainer is Use

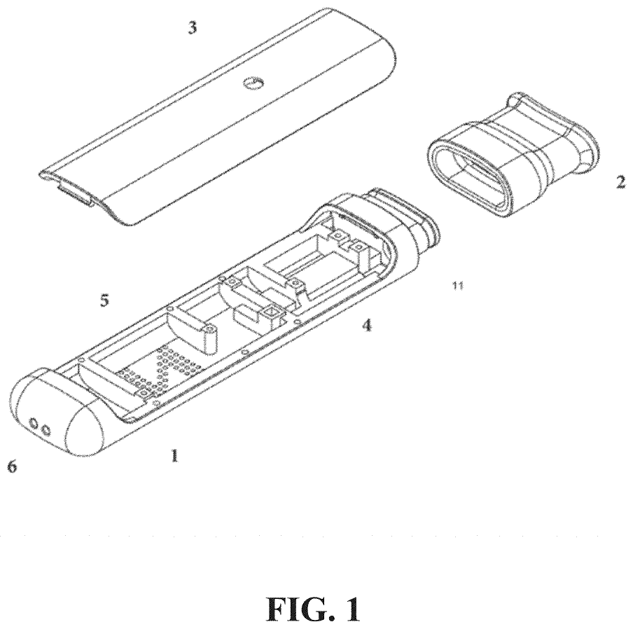

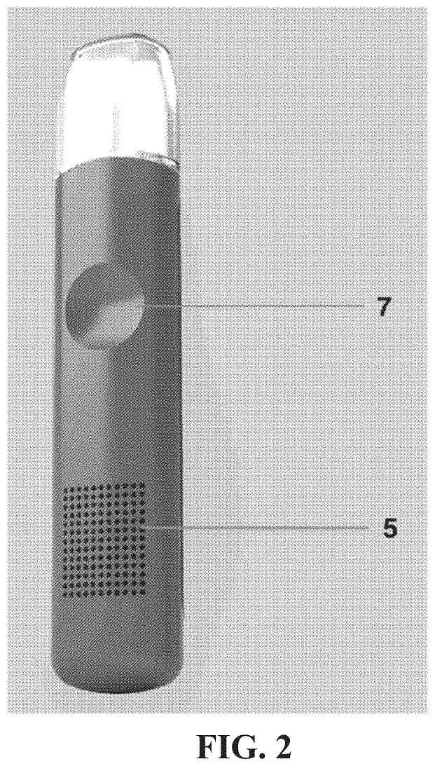

FIG. 1 is a diagram illustrating various examples embodiments of an exercise training and testing system that may include an RMT training device with a wireless connection to a smartphone with an RMT mobile application in accordance with the systems and methods described herein. FIG. 2 is another diagram illustrating various example embodiments of an exercise training and testing system that may include an RMT training device with a wireless connection to a smartphone with an RMT mobile application in accordance with the systems and methods described herein.

Referring mainly to FIGS. 1-2 in various example embodiments, the exercise training and testing system may include an RMT training device with, in some embodiments, a wireless connection (e.g., Bluetooth, Wi-Fi etc.) to a smartphone with an RMT mobile application. The mobile application on the smartphone (or iPad, computer, etc.) may be connected to the internet and to a central system, server, and database for storing and retrieving content and data live or on an archived basis.

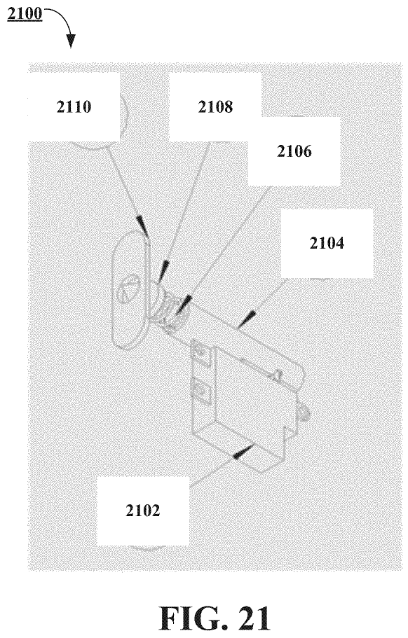

In different embodiments, the RMT trainer device comprises of a handheld housing frame 1, removable mouthpiece configurations 2, which may have a wide range of shapes for different exercise types; scuba mouthpiece and straight lip being two examples, a heart rate window 3, a power button 4, off gas vents 5, and charging port 6, a finger depression 7. The power button may serve as the stimulus to pair the hardware with the mobile phone, and in various embodiments the microcontroller unit (MCU) may also be awakened and paired with mobile applications without a button. The actuator may be defined as a combination of an electrical actuator (open loop or closed loop) or passive actuator that may be coupled with mechanical components which may include and not be limited to gearboxes, screw mechanisms, soft/rigid mechanical couplers. The actuated valve may be defined as an actuator that is coupled with a proportional area controlling valve mechanism including one or more of rigid/semi-rigid/flexible flaps, needles, poppets, spools, a pump, or a valve. An example actuated valve assembly, e.g., needle valve controller, is discussed with respect to FIGS. 21-23 below. This coupling may be single or multiple stages.

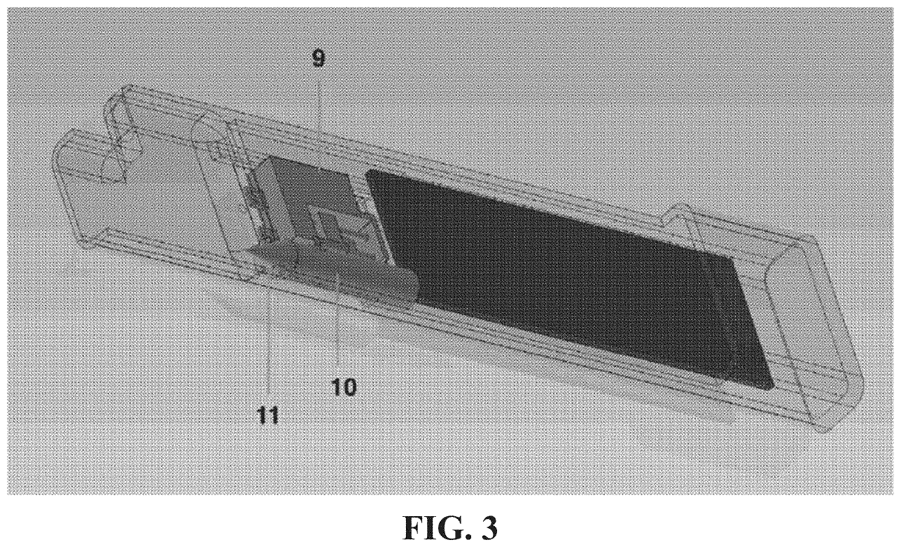

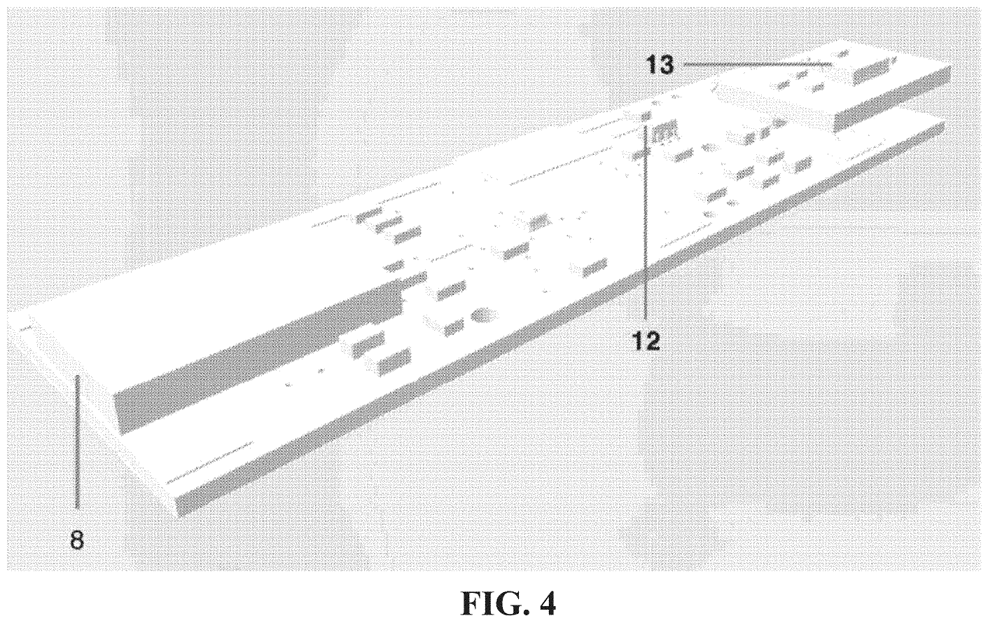

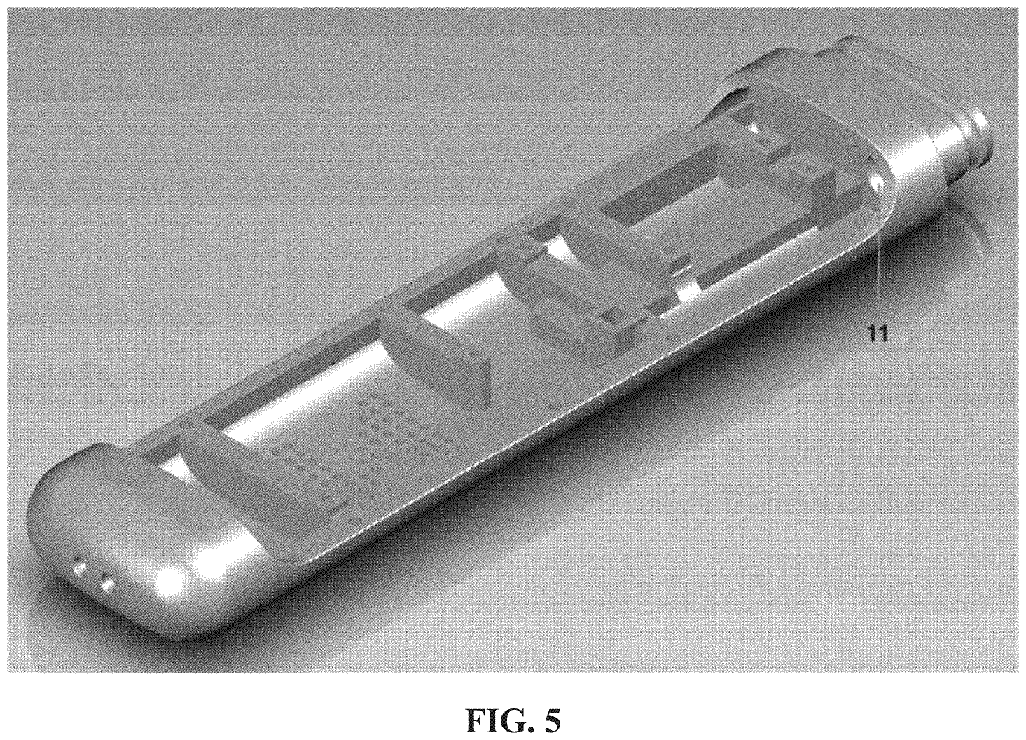

FIG. 3 is a diagram illustrating various examples embodiments of an inside of an RMT training device in accordance with the systems and methods described herein. FIG. 4 is another diagram illustrating various examples embodiments of an inside of an RMT training device include a pressure sensor, a heart rate monitor, digital and local storage on the device, a communication device, a camera, and a microphone in accordance with the systems and methods described herein. FIG. 5 is another diagram illustrating various examples embodiments of an inside of an RMT training device in accordance with the systems and methods described herein.

In various embodiments of the inside the RMT trainer device FIGS. 3-5, the MCU 8 drives the linear actuator 9 which in turn controls the resistance load 10.

Having an opening that may be controlled from 0.0 mm² area up to a certain mm². In various exemplary embodiments, the mechanisms may be controlled by electromechanical devices including electrical actuators such as to piezoelectric actuators, open loop and closed loop motors, solenoids coupled with mechanical components such as valves, gearboxes, screw mechanisms, flexible/rigid/semi flexible flaps, and any combination of these components herein. In this embodiment, the resistance load is designed by creating an airway opening 11 for the user to blow through with a maximum opening area and a flow which causes the buildup of a backpressure. To increase load and make muscle training more challenging, the actuator pushes the load controlling mechanism thereby reducing the area and creating the resistance mechanism 10.

In various embodiments, before the adaptive load adjustment occurs automatically, media in form of message, video, imagery, haptic vibration, or LED will be triggered to signal and guide the user if they are not performing the exercise correctly for their age-match threshold. To ensure maximal user compliance and to optimize training results, if the user is performing the exercise with the proper technique, but not within their baseline threshold, the mechanism will automatically actuate to allow more or less air to flow, and thus making the resistance higher or lower. Users have the option to deactivate this feature if they wish.

The digital hardware and software associated with the RMT trainer device includes various features that allow for tracking and training the user's biomarkers and muscles. In various embodiments, these digital features FIG. 4 include pressure sensors 12, heart rate monitor 13, digital and local storage on the device, communication, cameras, and microphones. The goal of using these sensors and their combined data capture, is to give users a better understanding of the complex interplay between heart, lungs, body and mind. To make all these data points effective to the user, in various embodiments of the device and accompanying mobile app, data communicated to the user is contextualized within a metric of the Breathability score called.

Breathability Fitness. This metric is an example of how the interplay of data from the heart and lungs could be codified and programmed into explainable machine learning models to easily communicate user's decline and progress toward their RMT fitness goals.

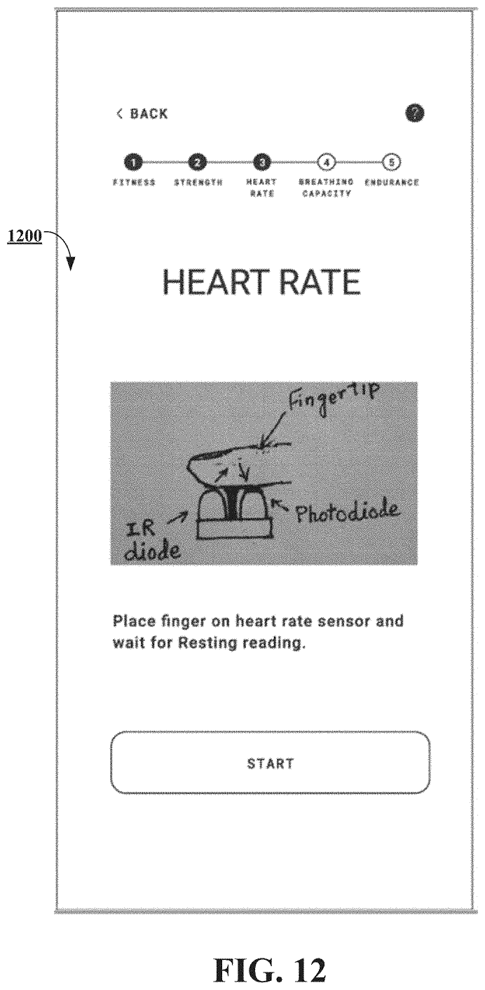

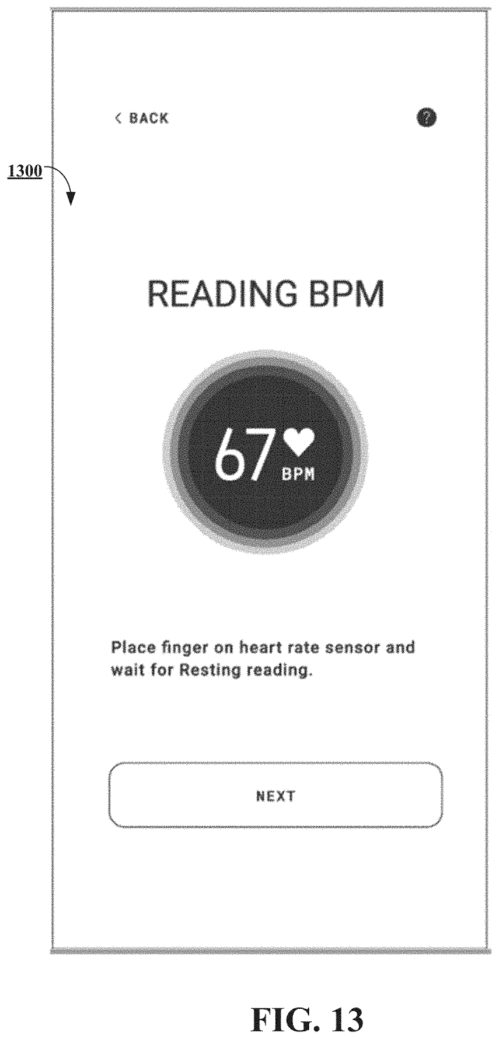

FIG. 12 is a diagram illustrating a breathability baseline test 1200 that may serve as a single, simple encompassing indicator of three central systems of the body in accordance with the systems and methods described herein. FIG. 13 is a diagram illustrating a breathability baseline test 1300 as part of an onboarding experience to build the user profile in accordance with the systems and methods described herein.

The Breathability baseline tests FIG. 12 serve as a single, simple encompassing indicator of three central systems of the body. The calculation of the Breathability score is defined in a later section. The test is a part of the onboarding experience to build the user profile FIG. 13. At the muscular and tissue level, oxygen consumption is examined by asking users about their physical activity (MVPA questionnaire) 14 and CO2 tolerance through an exhale test 15. Second, the vascular system is examined by measuring resting heart rate 16 and heart rate recovery 17. This helps assess how the lungs are delivering and removing waste from the body, the pump and plumbing. Finally, using the measurements of Maximal Inspiratory Pressure MIP 18, Maximal Expiratory Pressure (MEP) 19, Maximal Volume of Ventilation (MVV) 20 to help users understand their breathing capacity, and their ability to get air in and out.

This example of the position of the heart rate window 3 allows for users to have resting and recovery heart rate measured through their index finger during testing and training. Pressure sensors may be used to measure and calculate volume, flow, atmospheric pressure and user's respiratory pressure. A walk test may be administered using accelerometer sensors from the device and user's phone. For example, the walk test may be 1-10 minutes or longer. In one embodiment, the walk test may be a 6-minute walk test. All communication between the device and mobile or web application may be wireless accessed through Wi-Fi or Bluetooth, which is awakened when the power button is pressed.

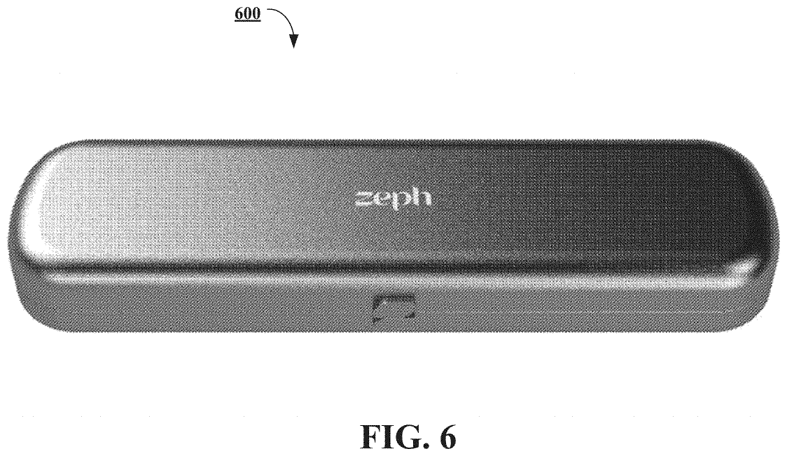

FIG. 6 is a diagram illustrating an RMT training device carrier 600 in accordance with the systems and methods described herein. In different embodiments, the RMT training device has a spit valve and an off gas which may sit in different places along the interior and exterior of the device's frame. The RMT trainer may be charged with a USBC charger or charge within its carrier 600 case FIG. 6.

How the Handheld Trainer is Operated.

The RMT training device comes preferably in two pieces: the body and the mouthpieces. In various exemplary embodiments, users will be guided through assembly to ensure the mouthpiece is properly attached to the body. As part of the assembly, users will receive a QR code or URL to download the mobile application. Once the application is downloaded, the user will be prompted with how to pair their handheld trainer with their phone via Bluetooth. Once this is set up, the handheld device will be connected to the user's phone for future use. When a user is ready to use their handheld device, they may wake up the device (which automatically pairs to phone after first pairing) by pressing the power button on the base of the handheld trainer. Designed guidelines such as finger depressions and ridging on the device guide the use to the proper handgrip. Prior to training, there is initial profile set up and onboarding FIG. 12 including taking the initial Breathability test. This allows for the user to be placed in the optimal program at the optimal level and threshold.

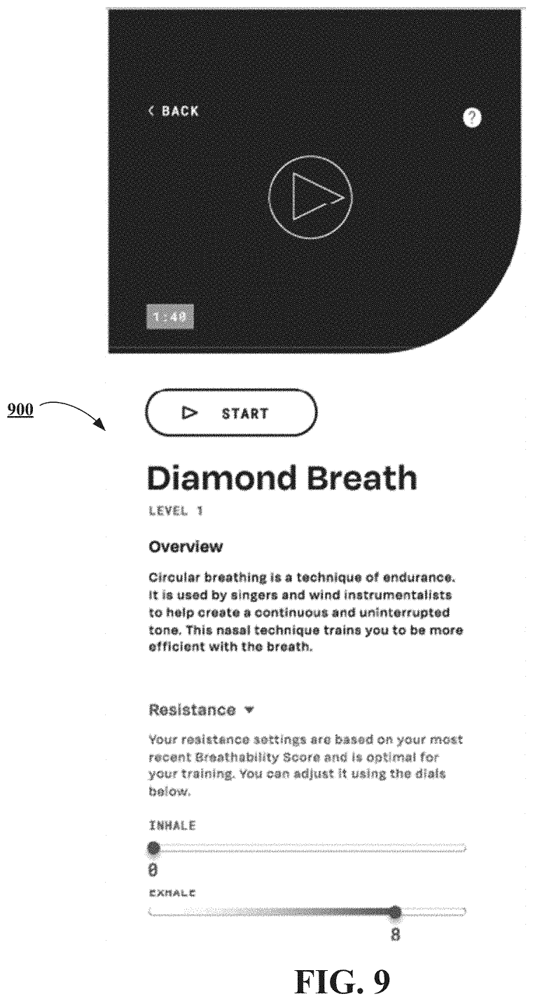

FIG. 9 is a diagram illustrating various examples of the embodiment of a mobile application having an example mobile interface 900 in which interacts with the RMT trainer device may communicate with the user interface via Bluetooth or Wi-Fi. Bluetooth (BLE) uses very little power to transfer data and instructions between the device and mobile phone in accordance with the systems and methods described herein.

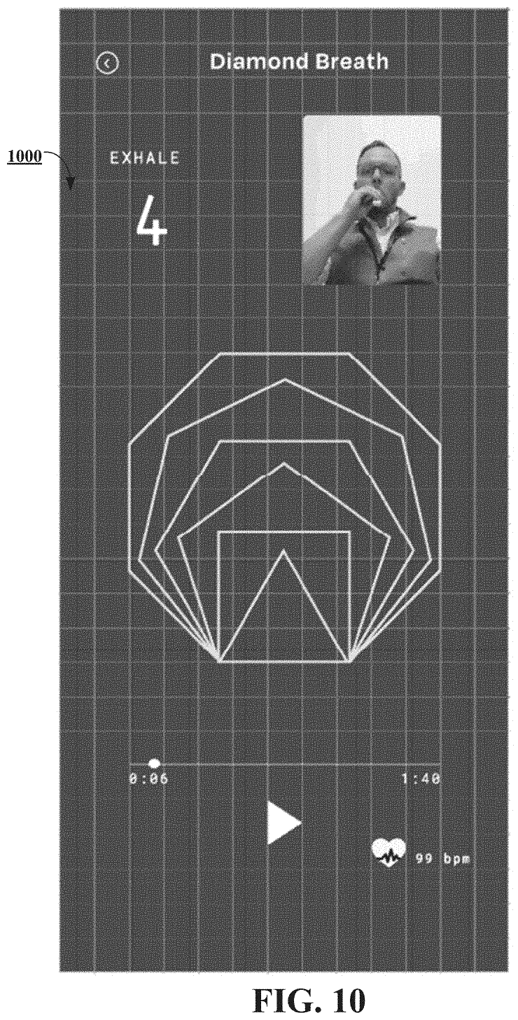

FIG. 10 is another diagram illustrating a mobile application screen 1000 in accordance with the systems and methods described herein. Once onboarding is complete and the user is ready to train, users will open an exercise on their mobile application FIGS. 9-10 and from this page, users will see their recommended resistance 21 based on their Breathability Score, goals and previous performance; however, users may override these suggested settings by adjusting the resistance on the app. In various embodiments, the position a user should be in for the exercise (sitting or standing, bending, etc.) will be suggested on the program screen for optimal performance of the exercise. Once the exercise is started, the user will feel a vibration in the handheld trainer to indicate the beginning of the exercise. In various embodiments, the exercises use lights and haptic vibrations in conjunction with the app screen to ensure proper user compliance, and to communicate feedback and instructions.

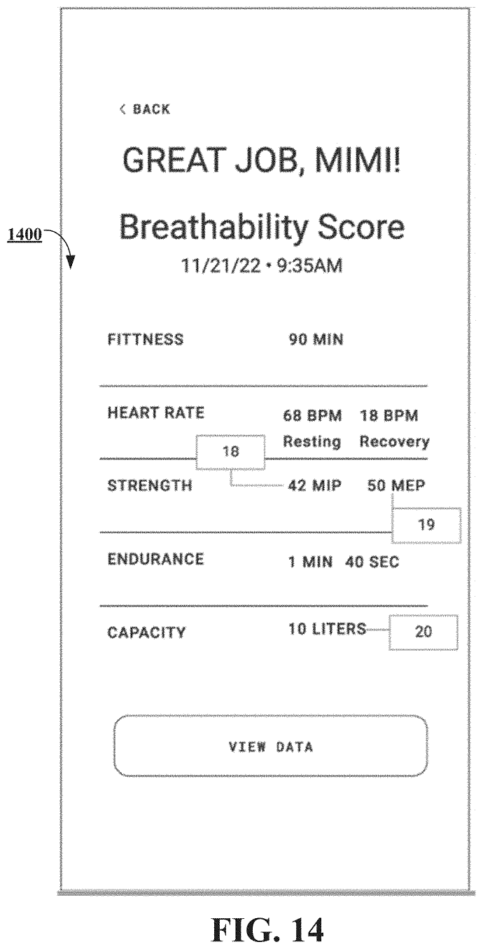

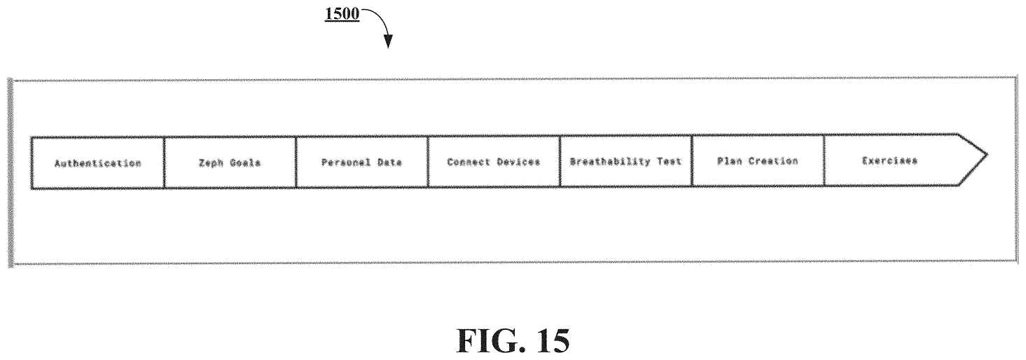

FIG. 14 is a diagram 1400 illustrating a breathability score in accordance with the systems and methods described herein. FIG. 15 is a diagram illustrating training 1500 in accordance with the systems and methods described herein.

Depending on the kind of program and level of the program, exercise trainings may take anywhere between 3 minutes and 15 minutes. For example, after training, a scorecard of results and metrics FIG. 15 may appear for quick highlights of how the user's exercise went for the user's lungs. An opportunity for the user to comment on their workout either writing freely (e.g., by dictation or typing) or using a selection of default comments may be used to document and save the user's summary screen. In various embodiments explainable machine learning may offer the user analysis of how this performance compared to previous performance.

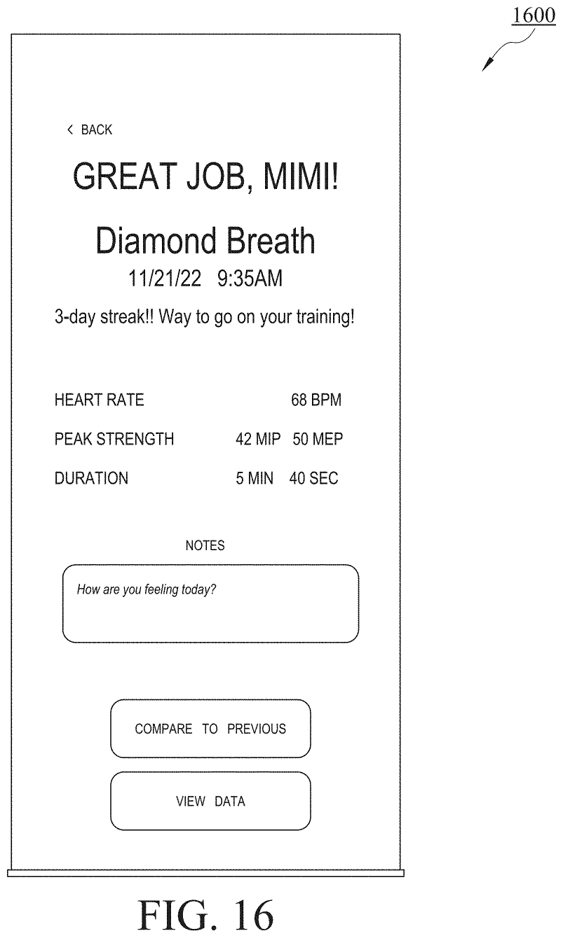

FIG. 16 is another diagram illustrating a mobile application 1600 in accordance with the systems and methods described herein. In various exemplary embodiments, a user may see what their breathing streak has been, with various accolades awarded for progress and consecutive logins and trainings. In group breath sessions, users may see what ‘level’ of breather users are. As an example, the ‘level’ is based on strength and power of someone's respiratory system, but also based on how dedicated and committed the user is to train. Additionally, exercises may be created to replicate the various levels of ability and targeted for different dysfunctions as exercises are performed in pulmonary rehabilitation. Once a user has gone through an exercise program to completion, the user may select from a library of programs created by the community's Advanced and Specialist users and Zeph Powersquad of professionals FIG. 16. Advanced and Specialist users might be existing breathwork coaches or practitioners. For instance, if world class swimmers, cyclists, runners or yoga instructors for mind and body training want to create training programs with the handheld device for their class, they may create the content and make it available on the app platform. In other embodiments, some exercises target the mind by increasing parasympathetic tone to reduce anxiety and downregulate sympathetic responses. Class lists will give users visibility into which exercise videos have been played the most and also favorited the most. Leaderboards provided through the mobile device provide users with a ranking of their RMT efforts compared to other users (e.g. anonymously, or otherwise) in their age group, class for improved breathing in swimming, cycling, running and other hobby sports selected. In other words, user's Breathability score and performance in training may be compared to other users of similar age, class and gender so that their respiratory performance may be understood generally or within the context of an environment they care about (cycling, or a certain dysfunction or cohort). The data provided to the leaderboard could be for live or archived classes. An individual's archived classes include the results of prior user's training activity which is presented on the leaderboard for comparison purposes. This historical log of exercises and user's performance may be revisited and done again, for example, whenever users want to try and improve on their past performance, they may individually play an archived class. As an example, a user's goal might be getting their heart rate lower or reaching a higher capacity or greater endurance than previous attempts. Additionally, as another embodiment, users may choose to do breath exercises together in live classes and classes that Advanced and Specialist user's create. Users have the ability to go back to archived group classes and uniquely compete with the leaderboard of top breathers.

How the Handheld Trainer Connects to a Network.

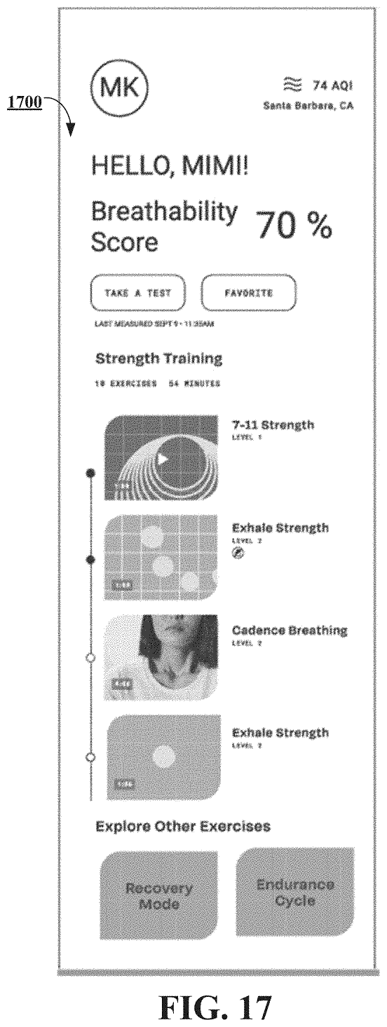

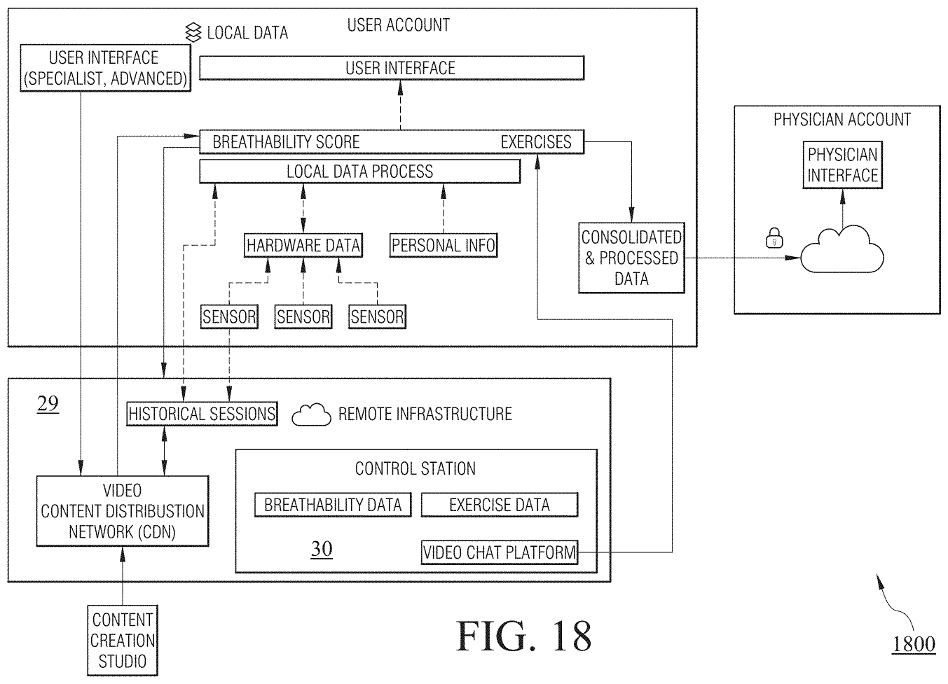

FIG. 17 is a diagram illustrating a summary page 1700 on a mobile application in accordance with the systems and methods described herein. FIG. 18 is a diagram illustrating that the system follows a well-established IoT paradigm 1800 whereby the device is paired with a user account connected via Bluetooth low energy (BLE) or Wi-Fi to an app on the mobile device in accordance with the systems and methods described herein.

Referring to FIGS. 17-18 the system follows a well-established IoT paradigm whereby the device is paired with a user account connected via Bluetooth low energy (BLE) or Wi-Fi to an app on the mobile device. During use, data collected by the device sensor is streamed real-time to the mobile and displayed on the screen for immediate visual feedback. This may be an overlay on top of a streamed class.

The data coming from the sensor is uploaded to the data warehouse in the cloud, so the user and their instructors may access it later and keep track of the progress.

If the mobile phone is connected to the internet during use, this data is uploaded immediately, otherwise if the user is exercising offline, the upload is delayed until the device is online. The most recent Breathability score and favorite exercises may be stored locally on the RMT trainer if the user chooses to activate local memory. Otherwise, all exercise content and sensor data may be saved to the cloud. If users want to simulate previous performance, they may access this training through historical sessions which may be stored on various databases 29. Historical and live sessions from the device may be collected and stored in control station 30. All exercise and Breathability data is also stored in the control station.

The burden of access control is on the mobile device. This is because it already supports a sophisticated system of biometric and password-based authentication. Once the user is authenticated on their mobile device, they may access their breathing device via BLE and their account online. The data warehouse communicates with user apps via a set of RESTful APIs.

User Mobile Interface

How the Handheld Trainer Works with Software.

In various exemplary embodiments, the resistance may be controlled remotely from the mobile or web application and different resistances may be selected for inspiratory and expiratory muscle training. Once resistance is set, the stepper motor 9 moves the load controlling mechanism 10 to a specific point to create the associated resistance load (cm H2O) on the user's airway. Depending on how a user is performing during exercises, this load resistance may automatically adjust placement to ensure the user is training in an optimal breathing zone and not straining or doing an exercise at too heavy of a load. The passive or active load controlling mechanism will actuate to maintain the prescribed pressure (cm H2O).

Capabilities of the Mobile Application

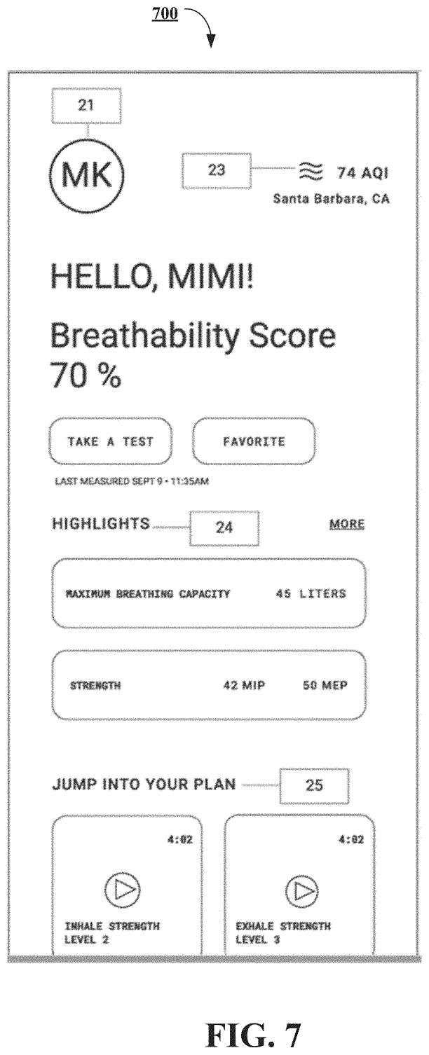

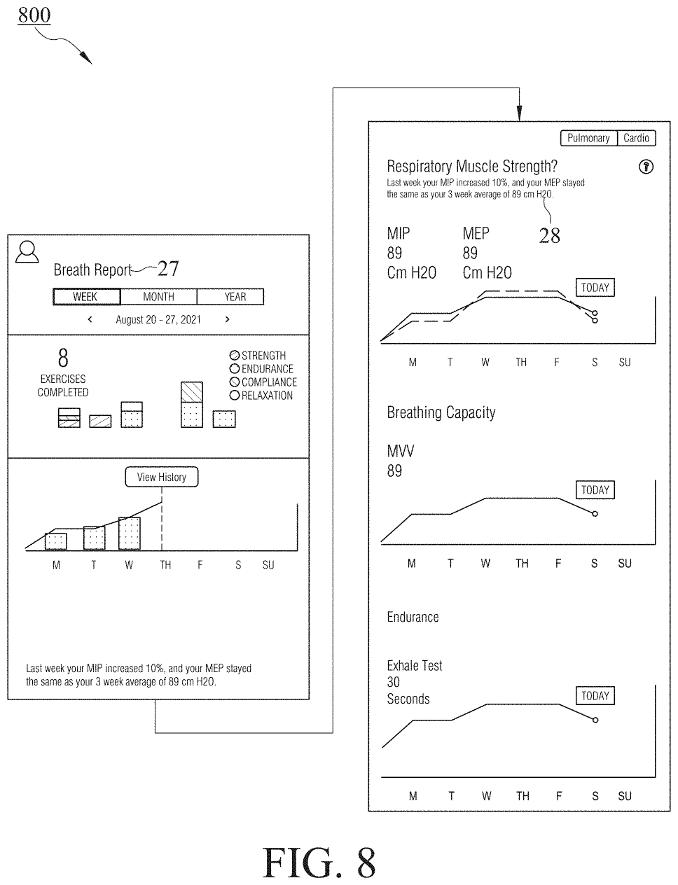

FIG. 7 is a diagram illustrating various examples of the embodiment of the mobile interface 700 which interacts with the RMT trainer device may communicate with the user interface via Bluetooth or Wi-Fi. Bluetooth (BLE) uses very little power to transfer data and instructions between the device and mobile phone in accordance with the systems and methods described herein. FIG. 8 is another diagram illustrating various examples of the embodiment of the mobile interface 800 which interacts with the RMT trainer device may communicate with the user interface via Bluetooth or Wi-Fi. Bluetooth (BLE) uses very little power to transfer data and instructions between the device and mobile phone in accordance with the systems and methods described herein.

Referring generally to FIGS. 7-9 in various examples of the embodiment of the mobile interface which interacts with the RMT trainer device may communicate with the user interface via Bluetooth or Wi-Fi. Bluetooth (BLE) uses very little power to transfer data and instructions between the device and mobile phone. The user interface may be run through a local program or application using local operating systems such as iOS or Android applications or via browser-based systems. Through the mobile interface FIG. 7, users will be able to login 21, logout of the system, view and add to their age-matched profile, access content, and review their past performances. Shortcuts to priority information such as Breathability Score 23, air quality 22, and their top highlighted metrics 24 may be found on the homepage FIG. 8. Deeper exploration of data is organized in the data page FIG. 8 in the breath report 27. Users may toggle between their data and performance from testing and training in different temporal views as well as metrics such as maximum, average, and total of pressure, flow, volume, capacity. These baselines will be compared to their age group, and to the average platform user 28. The mobile interface will also control the RMT trainer device haptic strength, resistance of exercises, power, and LEDs. Upon logging in, account set up and onboarding will lead to intake questions and profile creation that may be key to personalizing your RMT trainer device. A set of selected tests will be prompted for the user to go through the Breathability Baseline Test and analysis to properly assess the user's cardio-pulmonary state. Through these tests, an algorithm will assess the Breathability fitness of the user. Based on their Breathability score, a training plan will be recommended. Navigation between different screens: between homepage, plans, trainings, instructor content, breath blog, and data center is easily performed on the app.

The Breathability Tests and Score

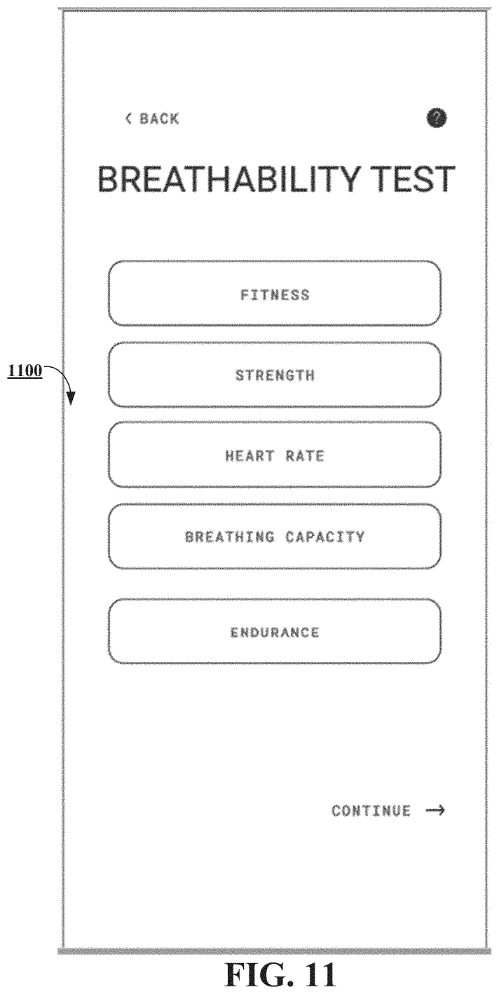

FIG. 11 is a diagram illustrating a start screen 1100 for a breathability test that may provide a single metric that weighs the different input data points at various weights to output a comprehensive Breathability fitness score accordance with the systems and methods described herein.

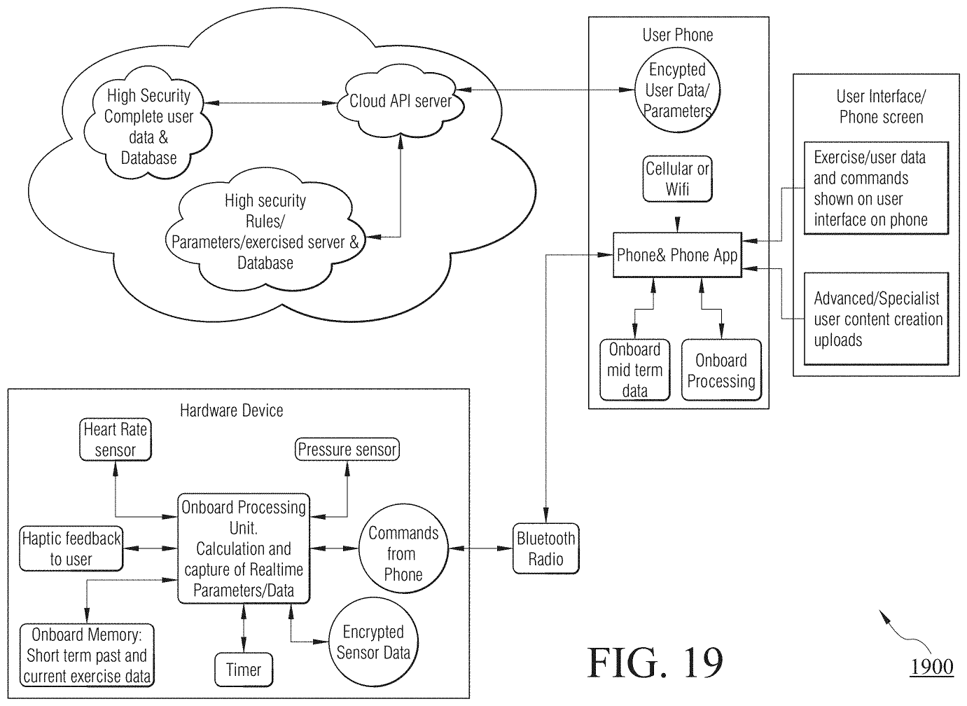

The Breathability score FIG. 11 is a single metric that weighs the different input data points at various weights to output a comprehensive Breathability fitness score. This score simplifies and contextualizes how much respiratory fitness and capacity one has after combining physiological and performance-based indicators of one's ability to breathe with respect to cardiac, pulmonary, and psychological demands of the respiratory system. An explanation of how the score is determined is explained later. The Breathability Score serves as a reference point for the user to understand their progress and serves as a key input data point for how machine learning models might modify trainings and make exercise recommendations such as increasing resistance or focusing on different respiratory muscles. In various embodiments, the RMT device is used to capture the user's Breathability baseline data, which comprises seven key data points as seen in FIG. 19. An example of a user's initial Breathability score and a subsequent score after training FIG. 15.

The collection of data biomarkers serves as an exemplary embodiment of one's Breathability fitness, which may be used to establish appropriate training intensities and progressions to optimize one's respiratory potential and serves as a baseline for care management.

Test of Maximum Inspiratory Pressure (MIP): the user puts on a nose clip, is instructed to fully expire and then perform a maximal inspiratory effort for at least 1.5 seconds. The peak negative pressure sustained for at least 1 second during that inspiratory maneuver is considered the maximal inspiratory pressure. The MIP is used to establish training percentages of the user's inspiratory muscle strength to suggest appropriate training parameters to improve the user's inspiratory muscle strength and endurance, depending on the program selected. The MIP represents 12.5% of the user's Breathability score.

Test of Maximum Expiratory Pressure (MEP): the user puts on a nose clip, is instructed to fully inhale and then perform a maximum expiratory effort for at least 1.5 seconds. The peak positive pressure sustained for at least 1 second during the expiratory maneuver is considered the maximum expiratory pressure. The MEP is used to establish training percentages of the user's expiratory muscle strength to suggest appropriate training parameters to improve the user's expiratory muscle strength and endurance, depending on the program selected. The MEP represents 12.5% of the user's Breathability score.

Breathing Capacity/Maximal Ventilatory Volume (MVV) Test: the user will be asked to perform maximal effort breathing for 15 seconds to measure the total volume of air exhaled during the 15 seconds. The MVV is used to estimate the user's minute ventilation, a test that may provide insight to respiratory muscle endurance and the ability to sustain activity levels over time. The MVV is a non-specific measure that is reduced in a variety of respiratory conditions. As the user's lung function improves with training, the MVV value is expected to improve over time. The MVV represents 25% of the user's Breathability score. At the beginning of the MVV measurement, the first maximal exhale may be captured to take note of the user's Forced Expiratory Volume (FEV) which measures how much air a person can exhale during a forced breath. The amount of air exhaled may be capture the amount during the first (FEV1), second (FEV2), and third seconds (FEV3) of the forced breath. In total, this first maximal exhale may be captured as the user's Forced Vital Capacity (FVC) which is the total amount of air exhaled during the FEV test.

Single Breath Exhale (SBE) Test/CO2 Tolerance Test: the user is instructed to fully inhale, and then slowly and gradually exhale for as long as they are able to without a stop in exhalation. This is a timed test which provides a data point for single breath hold capacity and the body's ability to tolerate rising CO2 levels, which may be a physiological trigger to signal the need for another breath. Individuals who are more resilient to stress, both physiologically and psychologically, may be able to tolerate more physical activity and better establish voluntary control over their respiratory system during activity. Performance on this timed test is tracked through the user's experience and is used to establish training parameters for breathing control and coordination exercises to train the user's ability to better control their breathing in response to the stresses of daily living and activity. The SBE represents 10% of the user's Breathability score.

Heart Rate Recovery (HRR) Test: the user will hold the RMT trainer to establish their resting heart rate and then perform a 3-minute bout of sustained light to moderate activity as well as a 6-minute walk test for patients. The user will be directed to sit down while maintaining a hold on the RMT trainer, which will calculate the heart rate over the 60 seconds immediately following activity. The heart rate at I minute following activity provides data on how well the user is able to recover following a bout of activity, this is an indicator of cardiopulmonary fitness and the body's ability to recover from stress. The 6-minute walk test (6WT) (or walk tests having other lengths of time) may follow guidelines set out by the American Thoracic Society. The 6WT sub-maximal exercise test used to assess aerobic capacity and endurance. The distance covered over a time of 6 minutes may be used as the outcome by which to compare changes in performance capacity, and may be measured using accelerometer in the user's phone which connects to the mobile app. An elevated HRR is an indicator of suboptimal cardiopulmonary fitness, and it is a data point that when tracked, may provide the user with appropriate training intensities and is a responsive data point when trained. The HRR represents 20% of the Breathability score.

Resting Heart Rate (RHR): the user will hold the RMT trainer to calculate a resting heart rate. Resting heart rate is a well-established indicator of cardiopulmonary fitness, systemic stress, and appropriateness for physical activity. The RHR represents 10% of the Breathability score.

Moderate-Vigorous Physical Activity (MVPA) minutes per week: the user will answer 2 questions to report the estimated time in minutes over the previous week that the user participated in moderate to vigorous amounts of physical activity. The RMT device and suggested training programs may address the foundational components of respiratory fitness, but the user is encouraged to integrate their trained capacity into both daily and recreational activities. These higher levels of respiratory fitness improve whole body conditioning and wellness, which when regularly performed serve to enhance the peripheral utilization and efficiency of oxygen consumption, thus reducing the demands on the respiratory system, making breathing more efficient. The MVPA represents I 0% of the Breathability score.

In a traditional setting, Pulmonary Function Test (PFT) may usually be performed as part of routine wellness screenings in hospitals and clinics, or as performance-based measures in a clinical setting when recovering from an injury or illness. Some embodiments make the data easily interpretable and applicable to its users to promote autonomy and self-efficacy in one's own health. An exemplary embodiment may use explainable Machine Learning (ML) to clearly explain to the user how their data has arrived at the present Breathability Score. This intelligent application allows for easily understandable reasoning for how some embodiments arrived at a given conclusion. The seven data points may be interpreted with weighted values compared to age and gender matched norms to determine the user's Breathability scores, a single metric to understand how their cardio-pulmonary performance compares with their peers and their own personalized potential. In various embodiments, the data captured from these tests may be performed as a baseline to establish an effective training regimen to enhance respiratory strength, endurance, and to optimize breathing control that is individualized to each user, regardless of their initial capabilities. For example, every ten sessions users must perform this baseline check-in to both track progress and establish new baselines. Explainable machine learning models will run to communicate changes in user's baselines and make modifications to their training programs. In various embodiments, these results may appear in the data center with different views of historical breathability scores.

Unique to hospital PFTs, these data points may be interpreted with different weighted values to determine the user's Breathability Score. As an exemplary embodiment, each data point could be weighed equally, or great weight could be attributed to the user's pulmonary data or more weight attributed to the user's fitness levels. The output score is interpreted out of 100 points and compared with the user's age-matched range. Unique to PFTs, some embodiments may suggest optimal training exercises to improve respiratory fitness based on baseline score. For instance, if a user performs their first Breathability test and receives a score of 72% and Explainability machine learning models indicate that their endurance is an area they could be improving most, their plan will queue up more endurance exercises at varying resistances. The optimal resistance determined in the plan will be automatically set in the RMT device at the start of an exercise, which may also be manually overruled if the user wants to overload or decrease.

This change in the RMT resistance will automatically be captured in the training data.

As mentioned, plan creation may be dependent on the user's Breathability score. The user's recommended progression may be calibrated to their unique profile and incoming Breathability baseline score. Once users have joined their personalized plan 25, they may follow a chronological playlist of classes with variable positions, frequencies, intensities, and durations. Users may choose to continue their plan or may choose from other category thumbnails offered. Certain Advanced and Specialist users who are accredited may collaborate to design unique trainings and teach or send these trainings to their own class. In this way, the platform may be adopted, and users may generate their own breathing exercises. Advanced and Specialist users may share their trainings with other users with an invitation, or the Advanced and Specialist users may add the exercises to the platform for others to explore. All classes may be liked so that the whole community may see who liked certain classes and how many people have done these classes to give a community view of enjoyment and ranking.

While training, elements of information about the user's performance may be displayed on the user interface FIGS. 10-11 including duration of training, elapsed time, time left, exercises being performed, heart rate, resistance load, and respiration pressure. After the class and training are finished, a data summary may be available for users to review and to see the highest pressure and total volume they were able to achieve during the class. Users may toggle the data page to see total trainings completed, historical performance, as well as a comparison to their personal best performances. This information may only be available to the user, not to anyone else, unless the user wants to send their score or baseline information to someone via email, text, or via Bluetooth.

The system may include cameras, speakers, or computer vision to ensure posture and proper compliance may be adhered to during at home trainings. If the user wants to redo an old exercise they enjoyed, they may, and the app will capture how many times a user re-does older exercises.

Users may also ‘Favorite’ trainings and quickly access these trainings from the homepage.

The content delivery to users and sharing of plans and exercises across the testing and training system is exemplary only and some embodiments of the present invention may be implemented in a number of different architectures. The network of pre-designed and pre-recorded exercises may be stored in the database server and may be sent instantly to the RMT trainer device and user interface. As users train and test with the RMT trainer device, their data may be sent to a backend server which includes one or more databases for storage. The user's local system (user interface) may be in constant communication with the servers either centralized or decentralized and the RMT device when the device is awake.

Content creation may occur in a variety of ways. Professional designing and animation, 3D cameras may be used to create 3D content, or professional trainers and therapists may record breathing exercises. In various exemplary embodiments, computer vision may be used in the user interface to observe the user's posture and diaphragm movement. Implementation of cameras, computer vision and audio recording may be used to help with user compliance. This data will be recorded and stored and distributed on the network of databases as well.

User Features and Resources

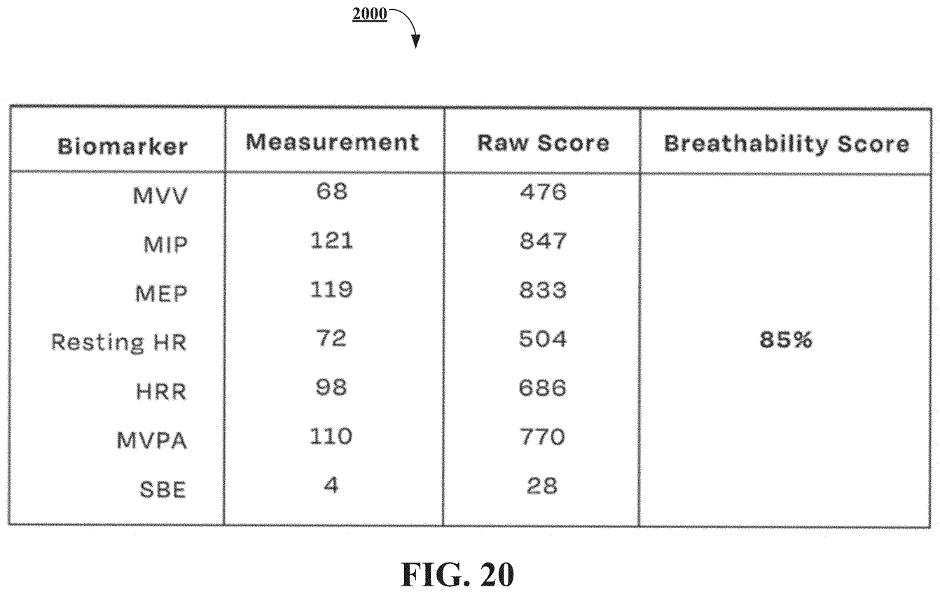

FIG. 19 is another diagram illustrating that the system follows a well-established IoT paradigm 1900 whereby the device may be paired with a user account connected via Bluetooth low energy (BLE) or Wi-Fi to an app on the mobile device in accordance with the systems and methods described herein. FIG. 20 is a diagram illustrating a summary of data and calculated scores 2000 that may be part of a breathability test on a mobile application in accordance with the systems and methods described herein.

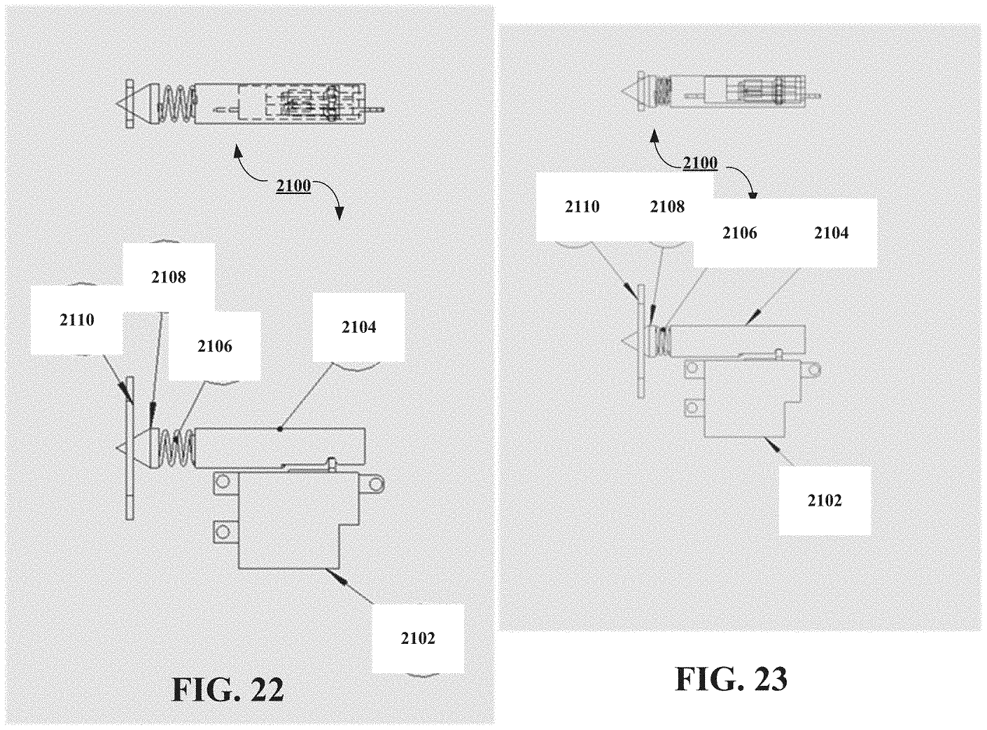

FIG. 21 is a diagram illustrating an example needle value 2100 in accordance with the systems and methods described herein. FIG. 22 is a diagram illustrating an example needle value 2100 in an open position in accordance with the systems and methods described herein. FIG. 23 is a diagram illustrating an example needle value 2100 in a closed position in accordance with the systems and methods described herein.

Referring to FIG. 21, the basic assembly of the needle valve 2100 controller, the basic components may include one or more of a controlled actuator 2102. The controlled actuator 2102 may include, but is not limited to electromechanical position, force, and velocity actuators. The basic assembly of the needle valve 2100 controller may include a needle valve base 2104, which may be attaches to the needle valve assembly (2,3,4) to the actuator. The basic assembly of the needle valve 2100 controller may also include a spring 2106 and a needle valve tip 2108. The needle valve tip 2108 may be a part that opens and closes the valve. The basic assembly of the needle valve 2100 controller may also include a chamber separating orifice 2110 (simplified for the drawing).

Functionality: Hybrid Valve

An example goal of the motorized system may be to have control over the position of the needle valve tip with respect to the orifice. The spring's maximum displacement (e.g., the biggest gap between the needle tip and body) may be mechanically limited and adjusted during calibration of the device in some embodiments. The position of FIG. 22 may be referred to as the “open” setting for the needle assembly (2104, 2106, 2108). When the needle is in “open” setting, the valve can be used as a regular needle valve controlling an orifice and/or opening to control the load for the user. The spring 2108 may be at its maximum opening and generally is not compressed in the open position.

The “closed” setting (FIG. 23) for the needle valve may be a variable setting with the variable being the distance between the needle valve base 2104 and the needle tip 2108. This distance may be controlled by having the actuator 2102 push the needle towards the orifice, compressing the spring. With precise control of the needle body position, the valve may become a passive pressure control valve with the needle tip moving back whenever the pressure in the high pressure chamber (to the right of chamber separating orifice 2110) becomes greater than a certain value, e.g., some predetermined value. This pressure setting value may be determined by the compression enforced on the spring 2106 by the actuator 2102.

The example mechanism may allow switching between two types of crucial valve types for different types of exercises. The first valve controls the open area of the orifice for indirect pressure and flow control by flow restriction (“open” valve setting) by positioning the needle specific positions with the actuator. The second valve allows for direct control of the pressure by creating a spring loaded pressure relief valve. Having both options can be advantageous in the variety of exercises and/or for the safety of the user by allowing direct pressure control when needed.

In various embodiments, users may use the system to manage their data, perform exercises, and set reminders. In app reminders may sync with calendars to remind users to track and train during their day or evening. There are several interactive features that allow users to engage with other users with game-like features; including a wide range of honors, awards and badges that may be won along the way when training. Creating structure around respiratory muscle training and strengthening gives one a pleasing orderly sort of feeling, similar to alphabetizing, to take life's random events and emotions and slot them into helpfully labeled shelves.

To train, users may join groups of competitions or support groups that Advanced and Specialist users in the community create to do together. Users may create pods that are open or require invitation. If a teacher wants to adopt the RMT trainer and create their own training for a class virtually or in person, they may submit a plan and make it available to a closed network or available on the platform for everyone to use.

One or more elements or aspects or steps, or any portion(s) thereof, from one or more of any of the systems and methods described herein may be combined with one or more elements or aspects or steps, or any portion(s) thereof, from one or more of any of the other systems and methods described herein and combinations thereof, to form one or more additional implementations and/or claims of the present disclosure.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A breathing exercise device, comprising:
a respiratory muscle training (RMT) device configured to communicatively connect to an electronic communication device, the RMT device further configured to:
measure biometric data of a user;
transmit the biometric data of the user to the electronic communication device; and
the biometric data transmitted while streaming at least one of instructional video content and instructional audio content to the electronic communication device.

2. The breathing exercise device of claim 1, wherein transmitting the biometric data of the user to the electronic communication device uses at least one of Wi-Fi, Bluetooth, and 5G communication standards.

3. The breathing exercise device of claim 1, wherein the electronic communication device includes at least one of a user screen and a speaker configured to receive the stream of the at least one of instructional video content and instructional audio content to the electronic communication device.

4. The breathing exercise device of claim 1, wherein the at least one of instructional video content and instructional audio content includes social competition capabilities.

5. The breathing exercise device of claim 1, the RMT device further comprising at least one sensor configured to measure biometric data of the user.

6. The breathing exercise device of claim 5, the at least one sensor comprising at least one of a pressure sensor or a heart rate monitor.

7. The breathing exercise device of claim 6, wherein the pressure sensor measures blood pressure.

8. The breathing exercise device of claim 1, further comprising at least one of a digital storage, a local storage on the device, a communication device, a camera, and a microphone.

9. The breathing exercise device of claim 1, further configured to create a tailored respiratory workout program with adaptive resistance training for the user based on the measure biometric data of the user.

10. The breathing exercise device of claim 1, further configured to allow users to review their data within a context of their community and other users with similar goals.
